(19) United States
(12) Patent Application Publication
(10) Pub. No.: US 2026/0294683 A1
(43) Pub. Date: Oct. 1, 2026

(54) INTRAOCULAR LENS (IOL) CUTTER FOR OPHTHALMIC SURGERY AND METHOD OF USING SAME

(71) Applicant: TONGJI HOSPITAL TONGJI MEDICAL COLLEGE OF HUST, Wuhan City (CN)

(72) Inventor: Ling JIANG, Wuhan City (CN)

(21) Appl. No.: 19/571,442

(22) Filed: Mar. 18, 2026

(30) Foreign Application Priority Data
Apr. 1, 2025 (CN) 202510404508.6

(51) Int. Cl. A61F 9/007; A61B 17/29; A61B 17/295; A61B 17/34; A61F 2/16

(52) U.S. Cl. CPC A61F 9/00736; A61F 2/16; A61B 2017/2938; A61B 17/295; A61B 17/3468

(57) ABSTRACT

The present disclosure belongs to the technical field of medical devices, and discloses an intraocular lens (IOL) cutter for ophthalmic surgery and a method of using the same. The cutter includes a barrel, wherein one end of the barrel is provided with a power assembly, and another end of the barrel is provided with a cutting assembly; the power assembly is drivingly connected to the cutting assembly via a first pulling rod and a second pulling rod; the cutting assembly includes a first cutting plate and a second cutting plate, wherein the first cutting plate is provided with a blade, and the second cutting plate is provided with a blade groove corresponding to the blade; an L-shaped plate is fixedly provided at an end of the second cutting plate proximate to the power assembly. By means of a mechanical structural design and a multi-linkage combined transmission inside the barrel.

### CROSS-REFERENCE TO RELATED APPLICATIONS

This application claims priority to Chinese Patent Application No. 202510404508.6, filed on Apr. 1, 2025, the content of which is incorporated herein by reference in its entirety.

### TECHNICAL FIELD

The present disclosure relates to the technical field of medical devices, and in particular to an intraocular lens (IOL) cutter for ophthalmic surgery and a method of using the same.

### BACKGROUND

In ophthalmic surgery, the precise removal and replacement of an IOL is a delicate operation requiring high technical skill. Modern ophthalmic surgery includes various types of IOL implantation procedures, such as phacoemulsification combined with IOL implantation (Phaco+IOL), scleral-sutured IOL implantation, and phakic IOL implantation (ICL) for refractive correction. These procedures aim to improve a patient's vision and ocular function through the implantation of an IOL.

However, after implantation, IOLs may face numerous complex medical challenges. Some patients may experience severe complications, such as partial fracture during folding and insertion of the IOL, dislocation of a suspended IOL, loss of transparency due to a proliferative membrane or pigment deposition on the lens surface, intraocular immune inflammation caused by the material, and refractory glaucoma or intraocular inflammation after ICL surgery. These conditions not only severely impair a patient's vision but may also threaten ocular health, thus necessitating the precise and minimally invasive removal of the original IOL.

Chinese patent document (Publication No. CN118453252B) discloses an intraocular foldable IOL grasping forceps, primarily used for removing an IOL previously implanted in a patient's eye; the forceps mainly comprise a main body, having an extended end and an operating end; a constraining sleeve, connected to and communicating with the extended end; a clamping mechanism, connected to the main body, having a clamped state and an open state, the clamping mechanism being convertible between the clamped state and the open state; a driving portion, rotatably disposed at the operating end of the main body and connected to the clamping mechanism, the driving portion being configured to drive the clamping mechanism to move towards or away from the constraining sleeve; the driving portion has a first rotation direction and a second rotation direction, wherein when the driving portion rotates in the first rotation direction, the clamping mechanism moves towards the constraining sleeve and can partially protrude from the constraining sleeve, and when the driving portion rotates in the second rotation direction, the clamping mechanism moves away from the constraining sleeve.

When an IOL is removed, an incision is required at the edge of the transparent cornea. Surgical instruments are inserted and withdrawn through this corneal incision to perform the IOL removal surgery. Several issues exist in the actual surgical operation:
1. In the above patent document, when the IOL texture becomes hard or the IOL size is relatively large, it is difficult to fold the IOL into the forceps for one-time removal, presenting significant limitations of the solution.
2. When removing the IOL in one piece using the entire grasping forceps, the hard IOL can easily scratch tissue, may scrape the corneal endothelium or even cause corneal endothelial detachment, and may also enlarge the size of the corneal incision, creating significant risks for subsequent recovery.
3. Cutting the IOL into smaller pieces with surgical scissors and then removing the pieces one by one with surgical forceps requires multiple passes of surgical instruments through the corneal edge incision. This frequent operation prolongs the surgery time and increases the probability of harming human tissue, dramatically increasing surgical safety risks.

### SUMMARY

In view of the deficiencies in the prior art, the present disclosure provides an IOL cutter for ophthalmic surgery and a method of using the same. By controlling a grip plate, a first pulling rod and a second pulling rod are driven to perform reciprocating linear movement. The first pulling rod drives a first cutting plate to open and close multiple times, causing a blade to cooperate with a blade groove to cut the IOL. The second pulling rod drives a roller of a transmission unit to rotate continuously, causing the roller and a driven roller to cooperate to transport the cut IOL segments towards the barrel. A pulling claw pushes the cut IOL into the barrel, and a pawl further pushes the IOL to an outlet of the barrel. The cutter of the present disclosure cuts the IOL to a suitable size for easy extraction. The cutter, upon a single entry into the cornea, can transport the cut IOL out through the barrel during the cutting process, avoiding multiple insertions and withdrawals of surgical instruments through the corneal incision, thereby improving efficiency and reducing surgical risks.

To achieve the above objectives, the present disclosure adopts the following technical solution.

An IOL cutter for ophthalmic surgery is provided. The cutter includes a barrel configured to collect an IOL, wherein an outlet is defined in the barrel; one end of the barrel is provided with a power assembly, another end of the barrel is provided with a cutting assembly, and the power assembly is drivingly connected to the cutting assembly via a first pulling rod and a second pulling rod; the cutting assembly includes a first cutting plate movably provided at an end of the barrel and a second cutting plate fixedly provided at the end of the barrel, wherein the first cutting plate is provided with a blade, and the second cutting plate is provided with a blade groove corresponding to the blade; an L-shaped plate is fixedly provided at an end of the second cutting plate proximate to the power assembly, wherein the L-shaped plate is mounted on the barrel via a rotating rod; the L-shaped plate is provided with a sliding sleeve in a direction perpendicular to the first cutting plate, wherein the sliding sleeve is connected to the second pulling rod via a first hinge member; an end of the L-shaped plate adjacent to the sliding sleeve is rotatably provided with a pulling claw; the second cutting plate is of a hollow structure, and a transmission unit is provided inside the second cutting plate; the first pulling rod passes through the second cutting plate to connect to the transmission unit, wherein the transmission unit includes a roller, and the roller passes through a top plate of the second cutting plate and extends outside; a clamping assembly is provided at an end of the barrel adjacent to the first cutting plate; when the power assembly drives the cutting assembly to perform cutting, the power assembly drives the clamping assembly to cooperate with the roller, allowing the cut IOL to enter the barrel.

Preferably, the transmission unit further includes a toothed plate, a first switching gear, a second switching gear, a first intermediate gear, a second intermediate gear, an idle gear, and an output gear, wherein the toothed plate is slidably disposed inside the second cutting plate, and a bottom of the toothed plate is fixedly connected to the first pulling rod; the first switching gear and the second switching gear are in meshing engagement with the toothed plate and spaced apart; the first intermediate gear is coaxially mounted with the first switching gear, wherein the first intermediate gear meshes with the output gear, and the output gear is coaxially arranged with the roller; the second intermediate gear is coaxially mounted with the second switching gear, wherein the second intermediate gear meshes with the idle gear, and the idle gear meshes with the output gear; and each of the first switching gear and the second switching gear is provided with a one-way bearing, and the two one-way bearings are configured to limit rotation in opposite directions.

Preferably, the clamping assembly includes a frame, wherein the frame is mounted on the rotating rod; a top of the frame is connected to an inner top of the barrel via a first tension spring; one end of the frame proximate to the roller is provided with a driven roller; another end of the frame is provided with a locking rod; the top of the frame is provided with a pressing rod, wherein the pressing rod and the first tension spring are both located between the rotating rod and the driven roller; and a locking assembly is provided above the locking rod.

Preferably, the locking assembly includes a T-shaped plate and a third tension spring; a T-shaped groove is defined in the inner top of the barrel; the third tension spring is disposed inside the T-shaped groove; the T-shaped plate is slidably mounted in the T-shaped groove; a bottom of the T-shaped plate is provided with an L-shaped groove corresponding to the locking rod; an inner opening of the L-shaped groove is provided with a rounded corner configured to facilitate sliding and pressing entry of the locking rod; and the bottom of the T-shaped plate is provided with a guide groove.

Preferably, a reset assembly includes a helical ridge wheel, a first rotating joint, a second rotating joint, a fourth tension spring, a limiting plate, and a thumbwheel; the second pulling rod is provided with the first rotating joint and the second rotating joint that are spaced apart; the helical ridge wheel and the limiting plate are fixedly provided on the second pulling rod between the two rotating joints and are spaced apart along the second pulling rod, wherein the helical ridge wheel is adjacent to the guide groove; the thumbwheel is sleeved outside the limiting plate; the thumbwheel is provided with a lever; a semi-annular groove is defined in the barrel around an outer periphery of the lever; and the fourth tension spring is connected between one side of the thumbwheel and an inner wall of the barrel.

Preferably, the power assembly includes an end cover, a handle, a grip plate, and a return spring; the end cover is detachably mounted on the barrel; ends of the first pulling rod and the second pulling rod that are proximate to the power assembly are fixedly provided with a connecting rod; a driving rod is fixedly provided on another side of the connecting rod; the return spring is sleeved on the driving rod, wherein the return spring is located between the connecting rod and a bottom plate of the end cover; the handle is fixedly provided on the end cover; the grip plate is arranged on the handle via a second hinge member; a limiting groove is defined in the grip plate; and the driving rod slidably passes through the bottom plate of the end cover and is then sleeved within the limiting groove.

Preferably, the end of the L-shaped plate adjacent to the sliding sleeve is connected to the pulling claw via a third hinge member, wherein a limit stop is provided on a side of the third hinge member proximate to the second cutting plate, and a second tension spring is connected between the limit stop and the pulling claw.

Preferably, a pawl is provided on the first pulling rod, wherein the pawl is located between the pulling claw and the outlet, and the pawl is configured to pull the IOL to transport the IOL to the outlet.

Preferably, edges of each of the first cutting plate, the second cutting plate, and the barrel are provided with rounded corners.

Preferably, a method of cutting an IOL in ophthalmic surgery using the cutter includes the steps of:
S1: Incising an edge of a cornea, inserting a head portion of the cutter into the cornea, and positioning the cutting assembly at a target location of the IOL; wherein, by an operator holding a handle and a grip plate and squeezing or releasing the grip plate, a driving rod is driven to perform reciprocating movement along an axial direction of the barrel, thereby driving a first pulling rod and a second pulling rod to perform reciprocating linear movement;
S2: Driving, by the second pulling rod, the first cutting plate to perform opening and closing movements, causing a blade disposed at a bottom of the first cutting plate to cooperate with a blade groove provided at a corresponding position on the second cutting plate, thereby cutting the IOL multiple times;
S3: Maintaining, by the first pulling rod through a gear train configuration of a transmission unit, a roller in continuous unidirectional clockwise rotation, thereby continuously transporting the cut IOL towards an inner portion of the barrel; wherein the unidirectional clockwise rotation of the roller is achieved by one-way bearings respectively provided on a first switching gear and a second switching gear, such that the roller maintains rotation in the same direction regardless of whether the first pulling rod moves from left to right or from right to left;
S4: Driving, by an L-shaped plate, a pulling claw to rotate when the first cutting plate and the second cutting plate open, such that the pulling claw abuts against and grasps the cut IOL falling from a top of the second cutting plate; and, when the first cutting plate and the second cutting plate subsequently move towards each other for a next cut, driving the pulling claw to push the IOL into the barrel; and
S5: Further transporting, by a pawl provided on the first pulling rod, wherein when the first pulling rod moves away from the second cutting plate, a tip of the pawl exerts a gripping force on the IOL, further pushing the cut IOL toward the outlet of the barrel.

Compared with the prior art, the present disclosure provides the following beneficial effects.
1. By means of a mechanical structural design and a multi-linkage combined transmission inside the barrel, the present disclosure achieves cutting an IOL within an eye into an appropriate size while simultaneously transporting the cut IOL into the barrel and then collecting the cut IOL through the outlet. Thus, the cutting, transporting, and collecting of the IOL are organically integrated into a single instrument, avoiding the drawback of multiple instrument insertions and withdrawals through the corneal incision in traditional surgery, thereby significantly reducing surgical risks and tissue damage. Secondly, the one-way bearing design of the transmission unit ensures that the roller rotates unidirectionally at all times, achieving precise and continuous transportation of the cut IOL. Furthermore, ingenious mechanisms such as the pulling claw and the pawl enable multi-step automatic operation within a confined surgical space to transport the IOL to the outlet. Moreover, the grip plate design of the power assembly is simple and ergonomic; a surgeon only needs to squeeze or release the grip plate to control the reciprocating movement of the first pulling rod and the second pulling rod, enabling quick and precise opening and closing of the first cutting plate for cutting, without requiring additional operations, and automatically completing the extraction of the cut IOL. The cutter can effectively handle IOLs that have become hard or are relatively large in size, overcoming the technical limitation of the prior art in removing hard lenses in one piece, and providing a more precise and minimally invasive technical solution for complex ophthalmic surgeries.
2. An operator can complete the complex cutting and transporting process simply by controlling a grip plate, making the operation more ergonomic. While the cutting assembly is operated to cut the IOL, the present disclosure enables the transmission unit, the clamping assembly, the pulling claw, the pawl, and the locking assembly to operate automatically through mechanical linkage, transporting the cut IOL, thereby realizing integrated operation of IOL cutting, transporting, and collecting, automatically switching between multiple operational states within the confined surgical space. The entire process relies on the automatic linkage of mechanical structures. The present disclosure drives the first pulling rod and the second pulling rod to perform reciprocating linear movement via the grip plate of the power assembly. The second pulling rod drives the first cutting plate to open and close for cutting, causing the blade to cooperate with the blade groove to cut the IOL. The first pulling rod drives the roller of the transmission unit to rotate continuously; during the reciprocating movement of the first pulling rod, the configuration of the transmission unit ensures that the roller always rotates in one direction, guaranteeing continuous movement of the cut IOL in a single direction. The roller cooperates with the driven roller to transport the cut small-sized IOL segments towards the barrel. The pulling claw grasps and pulls the cut IOL into the barrel, and the pawl provided on the first pulling rod further pushes the IOL towards the outlet of the barrel. The cutter of the present disclosure cuts the IOL to a suitable size for easy extraction. The cutter, upon a single entry into the cornea, can transport the cut IOL out through the barrel during the cutting process, avoiding multiple insertions and withdrawals of surgical instruments through the corneal incision, thereby improving efficiency, preventing incision enlargement or tissue collision and injury, and reducing surgical risks.

### BRIEF DESCRIPTION OF THE DRAWINGS

FIG. 1 is a perspective view of the overall structure of the cutter of the present disclosure;

FIG. 2 is a first schematic diagram showing the cutting assembly, the clamping assembly, and the locking assembly in cooperation of the present disclosure;

FIG. 3 is a second schematic diagram showing the cutting assembly, the clamping assembly, and the locking assembly in cooperation of the present disclosure;

FIG. 4 is a partial cross-sectional perspective view of the transmission unit of the cutter of the present disclosure;

FIG. 5 is a partial cross-sectional perspective view of the power assembly of the present disclosure;

### DETAILED DESCRIPTION OF THE EMBODIMENTS

The technical solutions in the embodiments of the present disclosure will be clearly and completely described below with reference to the embodiments of the present disclosure. Obviously, the described embodiments are only a part of the embodiments of the present disclosure, rather than all of the embodiments.

Contents not described in detail in this specification belong to the prior art known to persons skilled in the art. In the description of the present disclosure, it is to be understood that the terms “center”, “upper”, “lower”, “front”, “rear”, “left”, “right”, “vertical”, “horizontal”, “top”, “bottom”, “inner”, “outer”, “clockwise”, “counterclockwise” and the like indicate orientations or positional relationships based on the orientations or positional relationships shown in the drawings, and are used merely for convenience of description and to simplify the description of the present disclosure, and are not intended to indicate or imply that the referenced device or element must have a particular orientation, be constructed and operated in a particular orientation, and therefore shall not be construed as limiting the present disclosure. Furthermore, the terms “first”, “second”, “third” and the like are used merely for purposes of distinction, and shall not be construed as indicating or implying any relative importance.

### Embodiment 1

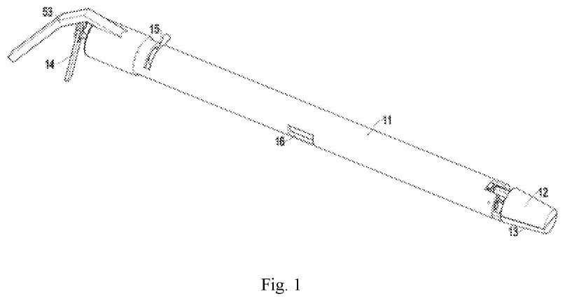

As shown in FIGS. 1-5, an IOL cutter for ophthalmic surgery is provided. The cutter includes a barrel 11 configured to collect an IOL, wherein an outlet 16 is defined in the barrel 11; one end of the barrel 11 is provided with a power assembly, another end of the barrel 11 is provided with a cutting assembly, and the power assembly is drivingly connected to the cutting assembly via a first pulling rod 27 and a second pulling rod 29; the cutting assembly includes a first cutting plate 12 movably provided at an end of the barrel 11 and a second cutting plate 13 fixedly provided at the end of the barrel 11, wherein the first cutting plate 12 is provided with a blade 18, and the second cutting plate 13 is provided with a blade groove 17 corresponding to the blade 18; an L-shaped plate 19 is fixedly provided at an end of the second cutting plate 13 proximate to the power assembly, wherein the L-shaped plate 19 is mounted on the barrel 11 via a rotating rod 36; the L-shaped plate 19 is provided with a sliding sleeve 20 in a direction perpendicular to the first cutting plate 12, wherein the sliding sleeve 20 is connected to the second pulling rod 29 via a first hinge member 25; an end of the L-shaped plate 19 adjacent to the sliding sleeve 20 is rotatably provided with a pulling claw 21; the second cutting plate 13 is of a hollow structure, and a transmission unit is provided inside the second cutting plate 13; the first pulling rod 27 passes through the second cutting plate 13 to connect to the transmission unit, wherein the transmission unit includes a roller 38, and the roller 38 passes through a top plate of the second cutting plate 13 and extends outside; and a clamping assembly is provided at an end of the barrel 11 adjacent to the first cutting plate 12; when the power assembly drives the cutting assembly to perform cutting, the power assembly drives the clamping assembly to cooperate with the roller, allowing the cut IOL to enter the barrel 11.

The present disclosure drives the first pulling rod 27 and the second pulling rod 29 to perform reciprocating linear movement via the grip plate 14 of the power assembly. The second pulling rod 29 drives the first cutting plate 12 to open and close repeatedly, causing the blade 18 to cooperate with the blade groove 17 to cut the IOL. The first pulling rod 27 drives the roller 38 of the transmission unit to rotate continuously; during the reciprocating movement of the first pulling rod 27, the configuration of the transmission unit ensures that the roller 38 always rotates in one direction, guaranteeing continuous movement of the cut IOL in a single direction. The roller 38 cooperates with the driven roller 37 to transport the cut small-sized IOL segments towards the barrel 11. The pulling claw 21 grasps and pulls the cut IOL into the barrel 11, and the pawl 28 provided on the first pulling rod 27 further pushes the IOL towards the outlet of the barrel 11. The cutter of the present disclosure cuts the IOL to a suitable size for easy extraction. The cutter, upon a single entry into the cornea, can transport the cut IOL out through the barrel 11 during the cutting process, avoiding multiple insertions and withdrawals of surgical instruments through the corneal incision, thereby improving efficiency, preventing incision enlargement or tissue collision and injury, and reducing surgical risks.

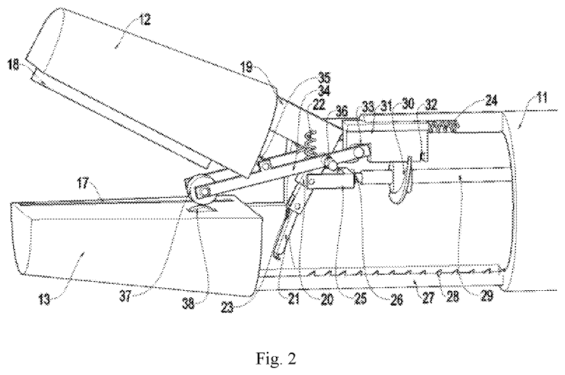

As shown in FIG. 2, the first pulling rod 27 extends from one end of the barrel 11 to the other end; specifically, the first pulling rod 27 extends from an end of the power assembly to an inner portion of the second cutting plate 13. The first pulling rod 27 may be disposed adjacent to an inner wall of the barrel 11, or may be disposed in a side wall of the barrel 11, extending through the wall of the barrel 11. The first pulling rod 27 is provided with a pawl 28. A through groove is defined in the inner wall of the barrel 11 within a movement region of the pawl 28, so as to facilitate the pawl 28 contacting the IOL that has entered the barrel 11 for subsequent transport to the outlet 16.

It should be noted that the blade 18 is located at a bottom of the first cutting plate 12 and is spaced apart from an edge thereof. The blade groove 17 is provided at a position corresponding to the blade 18, so as to facilitate cutting. The blade 18 is disposed inside, thereby avoiding damage to human tissue and ensuring operational safety.

Further, the transmission unit further includes a toothed plate 40, a first switching gear 41, a second switching gear 42, a first intermediate gear 46, a second intermediate gear 43, an idle gear 44, and an output gear 45, wherein the toothed plate 40 is slidably disposed inside the second cutting plate 13, and a bottom of the toothed plate 40 is fixedly connected to the first pulling rod 27; the first switching gear 41 and the second switching gear 42 are in meshing engagement with the toothed plate 40 and spaced apart; the first intermediate gear 46 is coaxially mounted with the first switching gear 41, wherein the first intermediate gear 46 meshes with the output gear 45, and the output gear 45 is coaxially arranged with the roller 38; the second intermediate gear 43 is coaxially mounted with the second switching gear 42, wherein the second intermediate gear 43 meshes with the idle gear 44, and the idle gear 44 meshes with the output gear 45; and each of the first switching gear 41 and the second switching gear 42 is provided with a one-way bearing, and the two one-way bearings are configured to limit rotation in opposite directions.

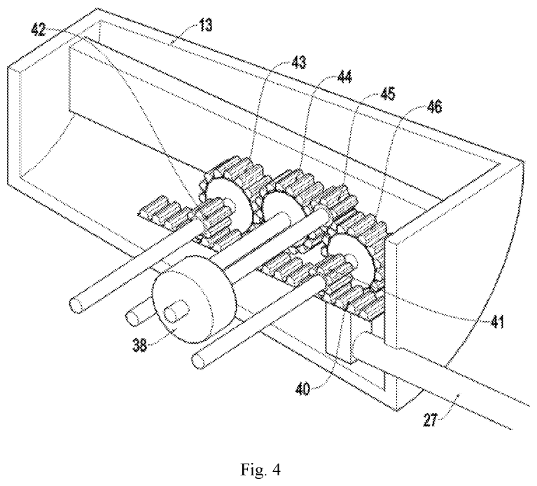

As shown in FIG. 4, a first one-way bearing is disposed between the first switching gear 41 and a rotating shaft on which the first switching gear 41 is mounted, and a second one-way bearing is disposed between the second switching gear 42 and a rotating shaft on which the second switching gear 42 is mounted.

When the first pulling rod 27 moves from right to left (as shown in FIG. 4), the first pulling rod 27 causes the toothed plate 40 to move from right to left, driving the second switching gear 42 to rotate clockwise. At this time, the first switching gear 41 rotates idly. The second intermediate gear 43, coaxially arranged with the second switching gear 42, rotates clockwise. The second intermediate gear 43 meshes with the idle gear 44 to drive the output gear 45, causing both the output gear 45 and the roller 38 coaxially arranged therewith to rotate clockwise. A linear velocity at a top of the roller 38 is directed toward an inner portion of the barrel 11. Simultaneously, the output gear 45 meshes with and transmits motion to the first intermediate gear 46, causing the first intermediate gear 46 and the rotating shaft on which the first intermediate gear 46 is mounted to rotate counterclockwise. Under an action of the first one-way bearing, the first switching gear 41 rotates idly.

When the first pulling rod 27 moves from left to right (as shown in FIG. 4), the first pulling rod 27 causes the toothed plate 40 to move from left to right, driving the first switching gear 41 to rotate counterclockwise. At this time, the second switching gear 42 rotates idly. The first intermediate gear 46, coaxially arranged with the first switching gear 41, rotates counterclockwise. The first intermediate gear 46 meshes with and drives the output gear 45, causing both the output gear 45 and the roller 38 coaxially arranged therewith to rotate clockwise. A linear velocity at a top of the roller 38 is directed toward the inner portion of the barrel 11. Simultaneously, the output gear 45 meshes with the idle gear 44 and then transmits motion to the second intermediate gear 43, causing the second intermediate gear 43 and the rotating shaft on which the second intermediate gear 43 is mounted to rotate clockwise. Under an action of the second one-way bearing, the second switching gear 42 rotates idly.

With the configuration of the transmission unit, regardless of whether the first pulling rod 27 moves from left to right or from right to left, the output gear 45 and the roller 38 coaxially arranged therewith rotate clockwise. This configuration further facilitates the roller 38 cooperating with the driven roller 37 to transport the IOL toward the inner portion of the barrel 11.

Still further, the clamping assembly includes a frame 34, wherein the frame 34 is mounted on the rotating rod 36; a top of the frame 34 is connected to an inner top of the barrel 11 via a first tension spring 22; one end of the frame 34 proximate to the roller 38 is provided with a driven roller 37; another end of the frame 34 is provided with a locking rod 39; the top of the frame 34 is provided with a pressing rod 35, wherein the pressing rod 35 and the first tension spring 22 are both located between the rotating rod 36 and the driven roller 37; and a locking assembly is provided above the locking rod 39.

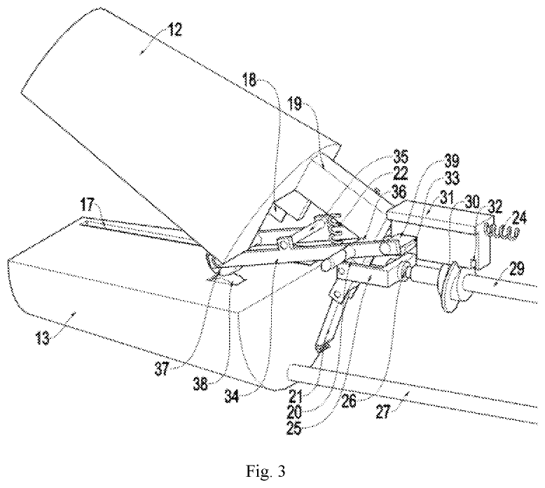

As shown in FIGS. 2 and 3, under the action of the first tension spring 22, an end of the frame 34 adjacent to the driven roller 37 is in a normally upward-tilted state and is not in contact with the roller 38. However, when the first cutting plate 12 and the second cutting plate 13 move toward each other to perform cutting, the L-shaped plate 19 on the first cutting plate 12 pushes downward against the pressing rod 35 at the top of the frame 34. This causes the driven roller 37 on the frame 34 to move downward toward the roller 38, and the locking rod 39 on the frame 34 to move upward. The locking rod 39 presses against the T-shaped plate 31 and then enters the L-shaped groove 33, thereby achieving a stable state under the action of the third tension spring 24. The driven roller 37 cooperates with the roller 38 to transport the cut IOL. When the clamped state of the clamping assembly needs to be released, the reset assembly is adjusted to disengage the locking rod 39 from the T-shaped plate 31, causing the driven roller 37 to move away from the roller 38.

Further, the locking assembly includes a T-shaped plate 31 and a third tension spring 24; a T-shaped groove is defined in the inner top of the barrel 11; the third tension spring 24 is disposed inside the T-shaped groove; the T-shaped plate 31 is slidably mounted in the T-shaped groove; a bottom of the T-shaped plate 31 is provided with an L-shaped groove 33 corresponding to the locking rod 39; an inner opening of the L-shaped groove 33 is provided with a rounded corner configured to facilitate sliding and pressing entry of the locking rod 39; and the bottom of the T-shaped plate 31 is provided with a guide groove 32.

As shown in FIGS. 2 and 3, two wings at a top of the T-shaped plate 31 are slidably disposed in the T-shaped groove. An end of the T-shaped plate 31 is connected to an inner portion of the T-shaped groove via the third tension spring 24. When the locking rod 39 presses upward against the T-shaped plate 31, the locking rod 39 enters the L-shaped groove 33 via a rounded corner of the L-shaped groove 33. After the locking rod 39 enters, the third tension spring 24 is in a stretched state, and upward and downward movement of the locking rod 39 is restricted.

Through a cooperative linkage among the second cutting plate 13, the L-shaped plate 19, the frame 34, and the locking assembly, the cutter of the present disclosure, upon initial use, can automatically position the IOL, which also facilitates subsequent transport. Within a confined space, multiple operational states can be automatically switched, saving space, simplifying manual operation, and improving work efficiency.

Still further, the reset assembly includes a helical ridge wheel 30, a first rotating joint 26, a second rotating joint 49, a fourth tension spring 47, a limiting plate 48, and a thumbwheel 15; the second pulling rod 29 is provided with the first rotating joint 26 and the second rotating joint 49 that are spaced apart; the helical ridge wheel 30 and the limiting plate 48 are fixedly provided on the second pulling rod 29 between the two rotating joints and are spaced apart along the second pulling rod 29, wherein the helical ridge wheel 30 is adjacent to the guide groove 32; the thumbwheel 15 is sleeved outside the limiting plate 48; the thumbwheel 15 is provided with a lever; a semi-annular groove is defined in the barrel 11 around an outer periphery of the lever; and the fourth tension spring 47 is connected between one side of the thumbwheel 15 and an inner wall of the barrel 11.

As shown in FIGS. 2, 3, and 5, the reset assembly is located between the first rotating joint 26 and the second rotating joint 49. A segment of the second pulling rod 29 on which the reset assembly is disposed is rotatable about an axis and is capable of transmitting force in an axial direction.

The thumbwheel 15 is provided with a lever. The lever is disposed in the semi-annular groove of the barrel 11. The semi-annular groove restricts axial displacement of both the lever and the thumbwheel 15, while also providing a slideway for the lever to rotate along the semi-annular groove. After rotation, the thumbwheel 15 is reset by the fourth tension spring 47. The thumbwheel 15 is slidably sleeved on the second pulling rod 29 and the limiting plate 48. The thumbwheel 15 is circumferentially fixed relative to the second pulling rod 29 but is capable of relative axial displacement, thereby facilitating reciprocating linear movement of the second pulling rod 29.

It should be noted that sliding fixing members may be disposed between each of the first pulling rod 27 and the second pulling rod 29 and the inner wall of the barrel 11, so as to facilitate stable movement of the first pulling rod 27 and the second pulling rod 29.

A helical protrusion is provided on an outer periphery of the helical ridge wheel 30. The helical protrusion is disposed corresponding to the guide groove 32 at the bottom of the T-shaped plate 31. The helical protrusion is a semi-helix, and the semi-helix corresponds to the semi-annular groove. When the thumbwheel 15 rotates in the semi-annular groove, the semi-helical protrusion rotates therewith, driving the T-shaped plate 31 to move and disengage from the locking rod 39, thereby completing resetting.

When the restriction of the locking assembly needs to be released, the thumbwheel 15 of the reset assembly is rotated, causing the second pulling rod 29 between the first rotating joint 26 and the second rotating joint 49 to rotate, thereby driving the helical ridge wheel 30 to rotate. When the helical protrusion enters the guide groove 32, the helical protrusion drives the T-shaped plate 31 to move away from the third tension spring 24, aligning a lower opening of the L-shaped groove 33 with the locking rod 39. Under the resetting action of the first tension spring 22, the end of the frame 34 where the driven roller 37 is located moves upward, and the end of the frame 34 where the locking rod 39 is located moves downward, thereby disengaging the locking rod 39 from the T-shaped plate 31 and releasing the locking state.

Further, the power assembly includes an end cover 52, a handle 53, a grip plate 14, and a return spring 51; the end cover 52 is detachably mounted on the barrel 11; ends of the first pulling rod 27 and the second pulling rod 29 that are proximate to the power assembly are fixedly provided with a connecting rod; a driving rod 50 is fixedly provided on another side of the connecting rod; the return spring 51 is sleeved on the driving rod 50, wherein the return spring 51 is located between the connecting rod and a bottom plate of the end cover 52; the handle 53 is fixedly provided on the end cover 52; the grip plate 14 is arranged on the handle 53 via a second hinge member 54; a limiting groove 55 is defined in the grip plate 14; and the driving rod 50 slidably passes through the bottom plate of the end cover 52 and is then sleeved within the limiting groove 55.

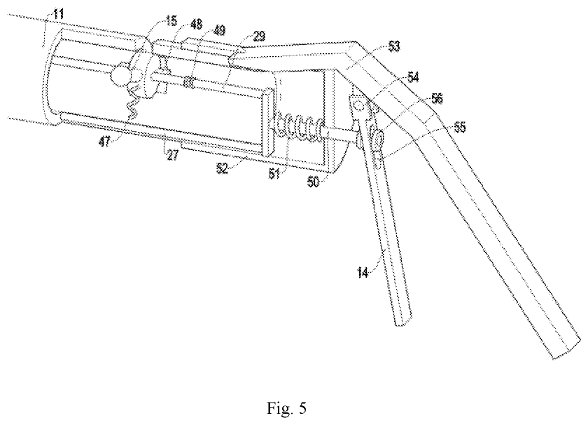

As shown in FIG. 5, when an operator holds the handle 53 and the grip plate 14 and squeezes or releases to switch, the driving rod 50 is driven to perform reciprocating movement along the axial direction of the barrel 11, thereby driving the first pulling rod 27 and the second pulling rod 29 to perform reciprocating movement.

The first pulling rod 27 drives the toothed plate 40 in the transmission unit to perform reciprocating linear movement. Through the switching and cooperation of the gear train, the roller 38 is caused to rotate unidirectionally, transporting the IOL toward the barrel 11. Simultaneously, the first pulling rod 27, via the pawl 28, also pushes the IOL into the barrel 11 and moves the IOL toward the outlet 16.

The second pulling rod 29 drives the first cutting plate 12 to repeatedly move closer for cutting, and also cooperatively drives the clamping assembly to move downward, facilitating smooth transport of the IOL.

Still further, the end of the L-shaped plate 19 adjacent to the sliding sleeve 20 is connected to the pulling claw 21 via a third hinge member, wherein a limit stop is provided on a side of the third hinge member proximate to the second cutting plate 13, and a second tension spring 23 is connected between the limit stop and the pulling claw 21.

As shown in FIGS. 2 and 3, under a normal state, due to the action of the second tension spring 23, the pulling claw 21 is aligned with an axial direction of the sliding sleeve 20.

When the first cutting plate 12 and the second cutting plate 13 open and move away from each other, the L-shaped plate 19 and the pulling claw 21 are driven to rotate together clockwise (as shown in FIG. 2) about the rotating rod 36. The pulling claw 21 moves toward the second cutting plate 13. At this time, the pulling claw 21 abuts against the IOL falling from the top of the second cutting plate 13, and the pulling claw 21 is forced to rotate counterclockwise (as shown in FIG. 2) about the third hinge member. A tip of the pulling claw 21 moves to a position adjacent to the top of the second cutting plate 13, contacting the IOL.

When the first cutting plate 12 and the second cutting plate 13 move toward each other, the L-shaped plate 19 and the pulling claw 21 are driven to rotate together counterclockwise (as shown in FIG. 2) about the rotating rod 36. The pulling claw 21 moves away from the second cutting plate 13. Simultaneously, under the action of the second tension spring 23 and restricted by the limit stop, the pulling claw 21 returns to alignment with the axial direction of the sliding sleeve 20 and continues rotating counterclockwise (as shown in FIG. 2) together with the sliding sleeve 20. At this time, the tip of the pulling claw 21 pulls the IOL to move toward the inner portion of the barrel 11, promoting transport of the cut IOL into the barrel 11.

The first cutting plate 12, the L-shaped plate 19, and the pulling claw 21 form a linkage, which both cuts the IOL and simultaneously pulls the cut IOL into the barrel 11, thereby integrating cutting, transporting, and collecting into one operation. This avoids multiple insertions and withdrawals of surgical instruments through the corneal incision, as in traditional procedures, which pose safety risks to the human eye.

Further, a pawl 28 is provided on the first pulling rod 27, wherein the pawl 28 is located between the pulling claw 21 and the outlet 16, and the pawl 28 is configured to pull the IOL to transport the IOL to the outlet 16.

As shown in FIG. 2, the pawl 28 is disposed on the first pulling rod 27 via a spring (not shown in the drawings). During transport of the IOL, the IOL covers the first pulling rod 27 and the pawl 28. When the first pulling rod 27 moves toward the second cutting plate 13, the tip of the pawl 28 is compressed into the first pulling rod 27, and the pawl 28 slides relative to the IOL. When the first pulling rod 27 moves away from the second cutting plate 13, the tip of the pawl 28 exerts a relatively large gripping force on the IOL, and the first pulling rod 27, via the pawl 28, pulls the cut IOL to move to the outlet 16.

### Embodiment 2

A method of cutting an IOL in ophthalmic surgery using the cutter includes the steps of:
S1: Incising an edge of a cornea, and inserting a head portion of the cutter into the cornea; wherein, by an operator holding a handle 53 and a grip plate 14 and squeezing or releasing the grip plate 14, a driving rod 50 is driven to perform reciprocating movement along an axial direction of the barrel 11, thereby driving a first pulling rod 27 and a second pulling rod 29 to perform reciprocating linear movement; during this process, a return spring 51 on a connecting rod provides a buffering and returning function;
S2: Driving, by the second pulling rod 29, the first cutting plate 12 to perform opening and closing movements, causing a blade 18 disposed at a bottom of the first cutting plate 12 to cooperate with a blade groove 17 provided at a corresponding position on the second cutting plate 13, thereby cutting the IOL multiple times;
S3: Maintaining, by the first pulling rod 27 through a special gear train configuration of a transmission unit, a roller 38 in continuous unidirectional clockwise rotation, thereby continuously transporting the cut IOL toward an inner portion of the barrel 11; wherein the unidirectional clockwise rotation of the roller 38 is achieved by one-way bearings respectively provided on a first switching gear 41 and a second switching gear 42, such that the roller 38 maintains rotation in the same direction regardless of whether the first pulling rod 27 moves from left to right or from right to left;
S4: Driving, by an L-shaped plate 19, a pulling claw 21 to rotate when the first cutting plate 12 and the second cutting plate 13 open, such that the pulling claw 21 abuts against and grasps the cut IOL falling from a top of the second cutting plate 13; and, when the first cutting plate 12 and the second cutting plate 13 subsequently move towards each other for a next cut, driving the pulling claw 21 to push the IOL into the barrel 11; this process precisely controls a movement trajectory of the pulling claw 21 through a third hinge member and a limit stop; and
S5: Further transporting, by a pawl 28 provided on the first pulling rod 27, wherein when the first pulling rod 27 moves away from the second cutting plate 13, a tip of the pawl 28 exerts a relatively large gripping force on the IOL, further pushing the cut IOL toward the outlet 16 of the barrel 11.

Through an ingenious mechanical design, the method achieves integrated operation of cutting, transporting, and collecting the IOL, significantly reducing a number of times surgical instruments enter and exit an incision of an eye, and improving surgical safety and efficiency.

The present disclosure discloses the technical concept of the present disclosure through the above embodiments, but the present disclosure is not limited to the above embodiments, meaning that the present disclosure does not have to rely on the above embodiments to be implemented. It should be understood by those skilled in the art that relevant improvements to the present disclosure fall within the protection scope and the disclosure scope of the present disclosure.

What is claimed is:

1. An intraocular lens (IOL) cutter for ophthalmic surgery, comprising a barrel configured to collect an IOL, wherein an outlet is defined in the barrel; one end of the barrel is provided with a power assembly, another end of the barrel is provided with a cutting assembly, and the power assembly is drivingly connected to the cutting assembly via a first pulling rod and a second pulling rod; the cutting assembly comprises a first cutting plate movably provided at an end of the barrel and a second cutting plate fixedly provided at the end of the barrel, wherein the first cutting plate is provided with a blade, and the second cutting plate is provided with a blade groove corresponding to the blade; an L-shaped plate is fixedly provided at an end of the second cutting plate proximate to the power assembly, wherein the L-shaped plate is mounted on the barrel via a rotating rod; the L-shaped plate is provided with a sliding sleeve in a direction perpendicular to the first cutting plate, wherein the sliding sleeve is connected to the second pulling rod via a first hinge member; an end of the L-shaped plate adjacent to the sliding sleeve is rotatably provided with a pulling claw; the second cutting plate is of a hollow structure, and a transmission unit is provided inside the second cutting plate; the first pulling rod passes through the second cutting plate to connect to the transmission unit, wherein the transmission unit comprises a roller, and the roller passes through a top plate of the second cutting plate and extends outside; and a clamping assembly is provided at an end of the barrel adjacent to the first cutting plate;
the transmission unit further comprises a toothed plate, wherein the toothed plate is slidably disposed inside the second cutting plate, and a bottom of the toothed plate is fixedly connected to the first pulling rod; a first switching gear and a second switching gear are in meshing engagement with the toothed plate and spaced apart;
a first intermediate gear is coaxially mounted with the first switching gear, wherein the first intermediate gear meshes with an output gear, and the output gear is coaxially arranged with the roller; a second intermediate gear is coaxially mounted with the second switching gear, wherein the second intermediate gear meshes with an idle gear, and the idle gear meshes with the output gear; and each of the first switching gear and the second switching gear is provided with a one-way bearing, and the two one-way bearings are configured to limit rotation in opposite directions;
the clamping assembly comprises a frame, wherein the frame is mounted on the rotating rod; a top of the frame is connected to an inner top of the barrel via a first tension spring; one end of the frame proximate to the roller is provided with a driven roller; another end of the frame is provided with a locking rod; the top of the frame is provided with a pressing rod, wherein the pressing rod and the first tension spring are both located between the rotating rod and the driven roller; and a locking assembly is provided above the locking rod;
the end of the L-shaped plate adjacent to the sliding sleeve is connected to the pulling claw via a third hinge member, wherein a limit stop is provided on a side of the third hinge member proximate to the second cutting plate, and a second tension spring is connected between the limit stop and the pulling claw; and
a pawl is provided on the first pulling rod, wherein the pawl is located between the pulling claw and the outlet, and the pawl is configured to pull the IOL to transport the IOL to the outlet.

2. The IOL cutter for ophthalmic surgery according to claim 1, wherein the locking assembly comprises a T-shaped plate and a third tension spring; a T-shaped groove is defined in the inner top of the barrel; the third tension spring is disposed inside the T-shaped groove; the T-shaped plate is slidably mounted in the T-shaped groove; a bottom of the T-shaped plate is provided with an L-shaped groove corresponding to the locking rod; an inner opening of the L-shaped groove is provided with a rounded corner configured to facilitate sliding and pressing entry of the locking rod; and the bottom of the T-shaped plate is provided with a guide groove.

3. The IOL cutter for ophthalmic surgery according to claim 2, wherein a reset assembly is provided on one side of the clamping assembly; the reset assembly comprises a helical ridge wheel, a fourth tension spring, and a thumbwheel; the second pulling rod is provided with a first rotating joint and a second rotating joint that are spaced apart; the helical ridge wheel and a limiting plate are fixedly provided on the second pulling rod between the two rotating joints and are spaced apart along the second pulling rod, wherein the helical ridge wheel is adjacent to the guide groove; the thumbwheel is sleeved outside the limiting plate; the thumbwheel is provided with a lever; a semi-annular groove is defined in the barrel around an outer periphery of the lever; and the fourth tension spring is connected between one side of the thumbwheel and an inner wall of the barrel.

4. The IOL cutter for ophthalmic surgery according to claim 1, wherein the power assembly comprises an end cover, a handle, a grip plate, and a return spring; the end cover is fixedly mounted on the barrel; ends of the first pulling rod and the second pulling rod that are proximate to the power assembly are fixedly provided with a connecting rod; a driving rod is fixedly provided on another side of the connecting rod; the return spring is sleeved on the driving rod, wherein the return spring is located between the connecting rod and a bottom plate of the end cover; the handle is fixedly provided on the end cover; the grip plate is arranged on the handle via a second hinge member; a limiting groove is defined in the grip plate; and the driving rod slidably passes through the bottom plate of the end cover and is then sleeved within the limiting groove.

5. The IOL cutter for ophthalmic surgery according to claim 1, wherein edges of each of the first cutting plate, the second cutting plate, and the barrel are provided with rounded corners.
